(19) United States
(12) Patent Application Publication
(10) Pub. No.: US 2026/0134945 A1
(43) Pub. Date: May 14, 2026

(54) METHODS FOR PREDICTING CELL-FREE PROTEIN SYNTHESIS

(71) Applicant: SYNVITROBIO, INC., San Leandro, CA (US)

(72) Inventor: Zachary Z. Sun, San Leandro, CA (US)

(21) Appl. No.: 19/119,295

(22) PCT Filed: Nov. 1, 2023
(86) PCT No.: PCT/US2023/036595

§ 371 Date: Apr. 8, 2025

Related U.S. Application Data
(60) Provisional application No. 63/421,784, filed on Nov. 2, 2022.

(51) Int. Cl. G16B 40/00; G06F 30/27

(52) U.S. Cl. CPC G16B 40/00; G06F 30/27

(57) ABSTRACT

The present invention provides methods and systems for determining the probability that a protein may be synthesized in a cell-free system. The invention provides a system that receives data comprising the properties of a protein to be synthesized, connects one or more databases comprising protein synthesis data, and calculates the probability of success for the protein to be synthesized. The inventions provide a prediction on whether a protein can or cannot be made. The system leverages protein databases to provide data on how proteins are made based on their digital sequence. The invention reduces unnecessary experimentation, synthesis costs, and further identifies success parameters to reduce the number of actions taken to get demonstrable synthesis.

WO

### FIELD OF THE INVENTION

The invention generally relates to methods and systems for protein synthesis.

### BACKGROUND

Proteins are vital to organisms and take part in virtually every process in an organism's cells. Unsurprisingly, functional proteins are of high demand in modern biotechnology. In producing proteins, host cells from one organism can be altered to produce proteins that would otherwise be produced from the cells of another organism. E. coli bacteria are a frequently used host for protein synthesis because of their relative simplicity and low cultivation costs.

Unfortunately, even in E. coli, synthesizing proteins is currently highly unpredictable.

Often, proteins formed are either insoluble and/or nonfunctional proteins. For example, success rates when trying to recombinantly express and purify soluble proteins in E. coli can be as low as 6%. As a result, no predicable method of protein synthesis exists. Rather, scientists must guess at synthesis ability by matching proteins from different hosts to strains, browsing prior literature, and trying different conditions to identify combinations that lead to production.

As a result, due to significant costs and burden of doing so, many proteins remain virtually un-synthesizable.

### SUMMARY

The present invention provides methods and systems for determining the probability that a protein may be synthesized in a cell-free system. The invention leverages the ability for cell-free system to provide synthesis data. The invention leverages this data to analyze whether to-be-synthesized proteins can be synthesized.

Notably, in cell-based systems, like E. coli, it is often difficult to scale reactions and collect consistent data tying protein sequences to outcome from cell-based systems. Cell-free systems, for example in vitro transcription-translation (TXTL) systems, allow for the rapid prototyping of genetic constructs in an environment that behaves similarly to a cell. In vitro systems can take only 8 hours and can scale to thousands of reactions a day, a multi-fold improvement over similar reactions in cells. The present invention leverages cell-free expression and the ability to conduct multiple protein expression runs at once, and collect data in a closed-loop format, to collect consistent data on protein synthesis outcomes. The present invention recognizes for the first that, that by increasing the richness of collected data, predictions en masse can be made from sequence to manufacturability.

The inventions provide a prediction on whether a protein can or cannot be made. The system leverages protein databases to provide data on how proteins are made based on their digital sequence. The invention reduces unnecessary experimentation, synthesis costs, and further identifies success parameters to reduce the number of actions taken to get demonstrable synthesis.

Aspects of the invention provide a system for determining the probability of cell-free synthesis of a protein. The system may comprise a computing device comprising a hardware processor coupled to memory containing instructions executable by the processor. The processor causes the system to receive data comprising the properties of a protein to be synthesized, connect to one or more databases comprising protein synthesis data, calculate the probability of success for the protein to be synthesized, and provide the calculated probability of success for the protein to be synthesized.

The one or more databases may include protein synthesis data for proteins previously processed through the system. The properties of the protein to be synthesized may be analyzed to calculate a property of success for the protein to be synthesized. For example, the properties of the protein analyzed can include (i) transmembrane helices, (ii) disulfide bonds, (iii) tryptophan residues, (iv) cysteine residues, (v) percent disorder, (vi) protein size, and (vii) any combination thereof. The properties of the protein for analysis may also not be readily identifiable to a biochemical trait but may be analyzed to calculate the probability of success for the protein to by synthesized. For example, the property may be statistical significance on a protein assay or test, or regulator of completement activation (RCA).

Advantageously, the system may be a closed loop-system. For example, the system may be adapted to update the one or more databases to include protein synthesis data for proteins processed through the system. The system may iteratively update the calculating step upon receiving additional protein synthesis data.

The one or more databases may comprise publicly available databases comprising protein structure and/or synthesis information. For example, the publicly available information platforms may be selected from a group consisting of: UCSB Protein Data Bank (PDB), National Library of Medicine (NLM), GenBank, Reference Sequence (RefSeq), UniProt, AlphaFold, or Expasy.

The Protein Data Bank (PDB) is an open access digital resource providing access to 3D structure data for large molecules, including proteins, provided by the Research Collaboratory for Structural Bioinformatics (RCSB). GenBank is a public database of known nucleotide and protein sequences, built and distributed by the National Center for Biotechnology Information (NCBI), a division of the National Library of Medicine (NLM). The Reference Sequence (RefSeq) database is an open access collection of publicly available nucleotide sequences (DNA, RNA) and their protein products. RefSeq was also built by National Center for Biotechnology Information (NCBI). UniProt is a freely accessible database of protein sequences and functional information, many entries being derived from genome sequencing projects. It contains information about the biological function of proteins derived from the research literature. It is maintained by the UniProt consortium, which consists of several European bioinformatics organisations. AlphaFold is a deep learning artificial intelligence program developed by DeepMind Technologies, headquartered in Longon, UK. AlphaFold performs predictions of protein structure. Expasy is an online bioinformatics resource operated by the Swiss Institute of Bioinformatics. Expasy provides access to over 160 databases and software tools and supports a range of life science and clinical research areas, including genomics, proteomics, structural biology, evolution, phylogeny, systems biology, and medical chemistry. The individual resources are hosted in a decentralised way by different groups of the SIB Swiss Institute of Bioinformatics and partner institutions.

The system may analyze information regarding reaction conditions for the protein to be synthesized to achieve the highest probability of success for protein synthesis based on the use of different reaction conditions. The reaction conditions that may be analyzed can include the (i) species for cell lysate to be used for in vitro synthesis, (ii) supplements for the in vitro synthesis, (iii) reaction temperature, (iv) duration of the reaction, (v) pH, (vi) solvents, (vii) technique for purification of synthesized protein, (viii) reagents for purification, (ix) concentration of DNA for in vitro synthesis, and (x) any combination thereof.

In aspects of the invention, the system may further provide recommendations for editing the sequence of the protein to be synthesized to increase the likelihood of success of synthesizing the protein or an analog to the protein. The system may further assign a protein family for the protein to be synthesized. For example, the protein family assignment may be based on the data available on Pfam database. Pfam is a database of protein families that includes their annotations and multiple sequence alignments generated using hidden Markov models.

Systems of the invention provide a probability of cell-free synthesis of a protein. For example, the probability for cell-free synthesis of the protein may be provided as standard, low risk, moderate risk, high risk, or unlikely. The probability for cell-free synthesis of the protein may also be provided quantitatively as a percentage, wherein 100% represents a high likelihood of success and 0% represents low likelihood of success.

Advantageously, the present invention also leverages machine learning systems, and in aspects of the invention the computing device comprise a machine learning system. The machine learning system may comprise a neural network.

Aspects of the invention also provide a method for determining the probability of cell-free synthesis of a protein. The method may comprise receiving data comprising the properties of a protein to be synthesized, connecting to one or more databases comprising protein synthesis data, calculating the probability of success for the protein to be synthesized, and providing the calculated probability of success for the protein to be synthesized.

As with systems of the invention, the one or more databases may include protein synthesis data for proteins previously analyzed with the method. The properties of the protein to be synthesized may be analyzed to calculate a property of success for the protein to be synthesized. For example, the properties of the protein analyzed can include (i) transmembrane helices, (ii) disulfide bonds, (iii) tryptophan residues, (iv) cysteine residues, (v) percent disorder, (vi) protein size, and (vii) any combination thereof. The properties of the protein for analysis may also not be readily identifiable to a biochemical trait but may be analyzed to calculate the probability of success for the protein to by synthesized. For example, the property may be statistical significance on a protein assay or test, or regulator of completement activation (RCA).

The method may further comprise updating the one or more databases to include protein synthesis data for proteins previously analyzed by the method. For example, the method may comprise iteratively updating the calculating step upon receiving additional protein synthesis data.

The methods may use databases that comprise publicly available databases comprising protein structure and/or synthesis information. For example, the publicly available information platforms may be selected from a group consisting of: UCSB Protein Data Bank (PDB), National Library of Medicine (NLM), GenBank, Reference Sequence (RefSeq), UniProt, AlphaFold, or Expasy.

The methods may analyze information regarding reaction conditions for the protein to be synthesized to achieve the highest probability of success for protein synthesis based on the use of different reaction conditions. The reaction conditions that may be analyzed can include the (i) species for cell lysate to be used for in vitro synthesis, (ii) supplements for the in vitro synthesis, (iii) reaction temperature, (iv) duration of the reaction, (v) pH, (vi) solvents, (vii) technique for purification of synthesized protein, (viii) reagents for purification, (ix) concentration of DNA for in vitro synthesis, and (x) any combination thereof.

The method may further comprise of the step of providing a recommendation for editing the sequence of the protein to be synthesized to increase the likelihood of success of synthesizing the protein or an analog to the protein. The method may further comprise the step of assigning a protein family for the protein to be synthesized. For example, the protein family assignment may be based on the data available on Pfam database.

Methods of the invention provide a probability of cell-free synthesis of a protein. For example, the probability for cell-free synthesis of the protein may be provided as standard, low risk, moderate risk, high risk, or unlikely. The probability for cell-free synthesis of the protein may also be provided quantitatively as a percentage, wherein 100% represents a high likelihood of success and 0% represents low likelihood of success.

Advantageously, the present invention also leverages machine learning systems, and in aspects of the invention methods may use a machine learning system. The machine learning system may comprise a neural network.

### BRIEF DESCRIPTION OF THE DRAWINGS

FIG. 1 is a flow chart for providing protein sequence-based predictions.

FIG. 2 is a picture of an exemplary output of the invention.

FIG. 3 is a graph of percent protein synthesis outcomes.

FIG. 4 is a graph of protein synthesis outcomes for each prediction score.

FIG. 5 is a graph of protein synthesis outcomes for moderate risk proteins.

FIG. 6 is a graph of protein synthesis outcomes based on Cystine (Cys) residues.

FIG. 7 is a graph of protein synthesis outcomes based on Tryptophan (Typ).

FIG. 8 is a graph of protein synthesis outcomes based on disordered percentage.

FIG. 9 is a graph of protein synthesis outcomes based on transmembrane helices (TMH). FIG. 10 is a graph of protein synthesis outcomes based on the number of disulfide bonds.

### DETAILED DESCRIPTION

The present invention provides methods and systems for determining the probability that a protein may be synthesized in a cell-free system. The invention provides a system that receives data comprising the properties of a protein to be synthesized, connects one or more databases comprising protein synthesis data, and calculates the probability of success for the protein to be synthesized. The inventions provide a prediction on whether a protein can or cannot be made. The system leverages protein databases to provide data on how proteins are made based on their digital sequence. The invention reduces unnecessary experimentation, synthesis costs, and further identifies success parameters to reduce the number of actions taken to get demonstrable synthesis.

The present invention leverages cell-free expression and the ability to conduct multiple protein expression runs at once, and collect data in a closed-loop format, to collect consistent data on protein synthesis outcomes. This is particularly advantageous, because despite the potential of cell-free systems, cell-free systems in particular need to be fine-tuned when used in different applications to achieve optimal results. Consistent data collected from cell-free systems enables a flywheel that provides increasingly predictive protein synthesis predictions for cell-free systems and their applications to achieve optimal results.

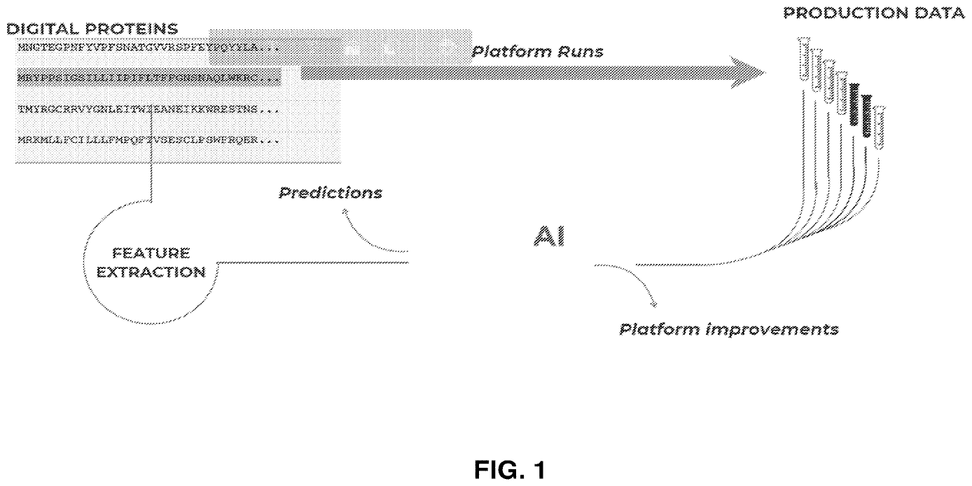

FIG. 1 is a flow chart for providing protein sequence-based predictions. Digital protein sequences are run through the system on the invention and a production provided. Protein synthesis is conducted, and actual production data correlated with the prediction. The production data is provided into the databases of the system to improve future predictions. The system extracts feature from the protein synthesis, including protein features and reaction features. The system provides an updated prediction for new proteins based on their digital protein sequence and similarity to proteins for which data is present.

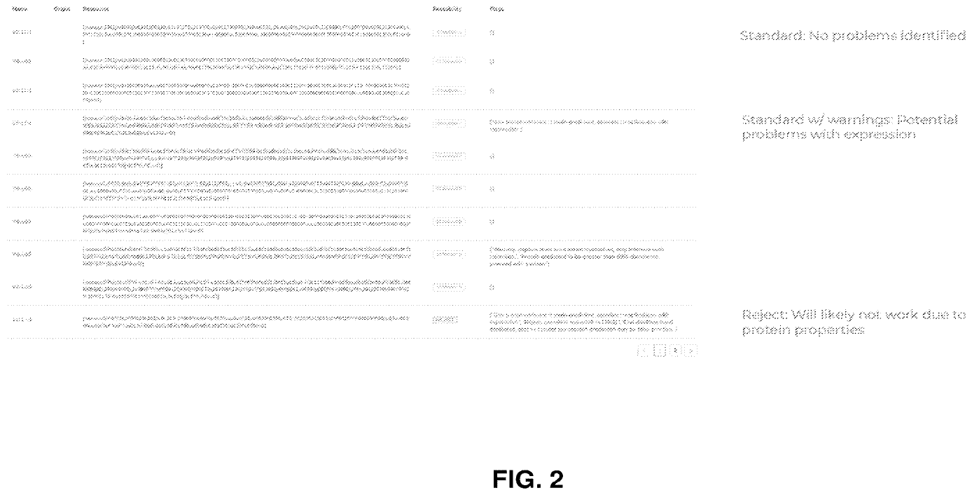

FIG. 2 is a picture of an exemplary output of the invention. For each sequence, a feasibility score is given for each digital protein sequence. Where there are no problems identified, the output provides a feasibility rating of “Standard.” The output also provides any flags present, even where a feasibility rating of “Standard” is given. For example, for V0.L04, the system identifies that “[‘One transmembrane domain predicted, possible complications with expression.’].” For V0.L08, the system provides the warning [‘“Warning: peptide does no contain tryptophan, may interfere with detection.’, ‘Protein predicted to be greater than 50% disordered, proceed with caution.’].” The fags identify potential problems that may arise with expression. The output also provides a feasibility rating of “Reject,” which indicates that synthesis will likely not work due to protein properties. For example, for V0.L10, the system identifies that [‘One transmembrane domain predicted, possible complication with expression.’, ‘Reject, protein is too small (<15 kD).’ ‘One disulfide bond predicted, proceed under assumption prediction may be false positive.’]

The invention can provide predication using known features of cell-free protein synthesis and assign protein features to expressibility from experiential learning. For example, related proteins or domains may be analyzing, for example via the Pfam database. Protein order/disorder data, membrane-regions, disulfide bonds, and protein size can be analyzed as features of cell-free protein synthesis.

The invention may also leverage the data lake from expressed proteins, mine public databases to expand datasets, and apply feature selection to discover influential factors and further improve the system with each protein synthesis reaction. Natural language processing may also be used to determine expression.

### Cell-Free Expression

In vitro transcription-translation cell-free systems have been developed which allow for the rapid prototyping of genetic constructs in an environment that behaves similarly to a cell. In vitro transcription and translation systems are systems that are to conduct transcription and translation outside of the context of a cell. This system is also referred to as “cell-free system”, “cell-free transcription and translation”, “TX-TL”, “TXTL”, “TX/TL”, “extract systems”, “in vitro system”, “ITT”, or “artificial cells.” Exemplary in vitro transcription and translation systems include purified or partially purified protein systems that are made from hosts, purified or partially purified protein systems that are not made from hosts, and protein systems made from a host strain that is formed as an “extract”. Extracts may include whole-cell extracts, nuclear extracts, cytoplasmic extracts, combinations thereof, and the like. Whole-cell extracts are also termed lysates herein. Lysates, and lysate systems, described herein, are intended to be non-limiting examples of extracts; where lysate is described herein, it is contemplated that other extracts, or extracts and protein combinations, may be used.

Cell-free systems may include a combination of cytoplasmic and/or nuclear components from cells. The components may include extracts, purified components, or combinations thereof. The extracts, purified components, or combinations thereof include reactants for protein synthesis, transcription, translation, DNA replication and/or additional biological reactions occurring in a cellular environment identifiable by a person skilled in the art.

Cell-free production biotechnology methods produce lysates from prokaryotic cells that are able to take recombinant DNA as input and conduct coupled transcription and translation to output enzymatically active protein. Cell-free systems take only 8 hours to express, rather than days to weeks in cells, since there is no need for cloning and transformation. They are also at least 10-fold cheaper to run than cells, and can be run in high-throughput as reactions are the equivalent of a reagent and used in a 384-well plate. Typical yields of prokaryotic systems are 750 μg/mL of GFP (30 μM). Cell-free systems with multiple organisms can be implemented and expression conducted at scales from 10 pi up to 10 mL.

Directions on how to make the extract component of cell-free systems, particularly lysates from E. coli, can be found in (Sun et al. 2013), which is hereby incorporated by reference herein in its entirety; other methods for producing a lysate are known to one of ordinary skill in the art. While this procedure is adapted for E. coli cell-free systems, it can be used to produce other cell-free systems from other organisms and hosts (prokaryotic, eukaryotic, archaea, fungal, etc.). Examples, without limitation, of the production of other cell-free systems include Streptomyces spp. (Thompson et al. 1984), Bacillus spp. (Kelwick et al. 2016), and Tobacco BY2 (Buntru et al. 2014), which are hereby incorporated by reference herein in their entireties. Exemplary processes for producing lysates involve growing a host in a rich media to mid-log phase, followed by washes, lysis by French Press and/or Bead Beating Homogenization and/or equivalent method, and clarification. A lysate that has been processed as such can be referred to as a “lysate”, or a “treated cell lysate”, and is a non-limiting example of an “extract.” Cells may be grown under anaerobic conditions and an extract may be prepared under anaerobic conditions. Any host cell may be used and analyzed by the predictive systems and methods of the invention. For example, any host cell may be analyzed for its compatibility with the synthesis of a protein.

Accordingly, in aspects of the invention, a “host” or “host cell” may be any prokaryotic or eukaryotic single cell (e.g., yeast, bacterial, archaeal, etc.) cell or organism. The host cell can be a recipient of a replicable expression vector, cloning vector or any heterologous nucleic acid molecule. Host cells may be prokaryotic cells such as species of the genus Escherichia or Lactobacillus, or eukaryotic single cell organism such as yeast. The heterologous nucleic acid molecule may contain, but is not limited to, a sequence of interest, a transcriptional regulatory sequence (such as a promoter, enhancer, repressor, and the like) and/or an origin of replication. As used herein, the terms “host,” “host cell,” “recombinant host” and “recombinant host cell” may be used interchangeably. For examples of such hosts, see Green & Sambrook, 2012,Molecular Cloning: A laboratory manual, 4th ed., Cold Spring Harbor Laboratory Press, New York, which are hereby incorporated by reference herein in their entireties.

One or more additives may be supplied along-side an extract to maintain gene expression. Contemplated additives include those tailored to replicate the in vivo expression and/or the metabolic environment of the lysate source organism, e.g., redox buffering agents, phosphate potential buffering agents, customized energy regeneration systems, native ribosomes, chaperones, species-specific tRNAs, pH buffering, metals (such as Magnesium and Potassium), osmoregulatory agents, gas concentrations; [O2], [CO2], [N2], sugars, maltose, starch, maltodextrin, glucose, glucose-6-phosphate, fructose-1,6-biphosphate, 3-phosphogly cerate, phosphoenolpyruvate, pyruvate kinase, pyruvate dehydrogenase, pyruvate, acetyl phosphate, acetate kinase, creatine kinase, creatine phosphate, glutamate, amino acids, ATP, GTP, CTP, UTP, ADP, GDP, CDP, UDP, AMP, GMP, CMP, UMP, folinic acid, spermidine, putrescine, betaine, DTT, TCEP, b-mercaptoethanol, TPP, FAD, FADH, NAD, NADH, NADP, NADPH, oxalic acid, CoA, glutamate-salts, acetate-salts, cAMP, native polymerases, synthetic polymerases, phage polymerases, temperature regulation conditions. A review of optional additives can be found in (Chiao et al. 2016), which is hereby incorporated by reference herein in its entirety. Optional additives may also include components that assist transcription and translation, such as phage polymerases, T7 RNA polymerase (RNAP), SP6 phage polymerase, cofactors, elongation factors, nanodiscs, vesicles, and antifoaming agents. Optional additives may also include additives to protect DNA, such as, without limitation, gamS, Ku, junk DNA, DNA mimicry proteins, chi site-DNA, or other DNA protective agents.

The reaction may include more than 0.1% (w/v) of crowding agent. Macromolecular crowding refers to the effects of adding macromolecules to a solution, as compared to a solution containing no macromolecules. Such macromolecules are termed crowding agents. A contemplated crowding agent may be from a single source, or may be a mix of different sources. The crowding agent may be from varied sizes. The crowding agents may include polyethylene glycol and its derivatives, polyethylene oxide or polyoxyethylene.

An energy recycling and/or regeneration system drives synthesis of mRNA and proteins by providing ATP to a system and by maintaining system homoeostasis by recycling ADP to ATP, by maintaining pH, and generally supporting a system for transcription and translation. A review of energy recycling systems can be found in (Chiao et al. 2016), which is hereby incorporated by reference herein in its entirety. Examples, without limitation, of energy recycling and/or systems that can be used include Glycerate 3-phosphate (3-PGA) (Sun et al. 2013), creatinine phosphate/creatinine kinase (CP/CK) (Kigawa et al. 1999), PANOx (Kim & Swartz 2001), and glutamate (Jewett & Swartz 2004). Other recycling and/or systems include those that can regenerate redox potential ([NAD(P)H]/[NAD(P+)]). An example of redox recycling is described in (Opgenorth et al. 2014). Recycling and/or systems can utilize innate central metabolism pathways from the host (for example, glycolysis, oxidative phosphorylation), externally supplied metabolic pathways, or both.

The in vitro transcription and translation system may include one or more nucleic acids. The nucleic acid may include DNA, RNA, or combinations thereof. A DNA may be supplied that that can produce a protein by utilizing transcription and translation machinery in the extract and/or additions to the extract. This DNA may have regulatory regions, such as under the OR2-OR1-Pr promoter (Sun et al. 2013), the T7 promoter or T7-lacO promoter, along with a RBS region, such as the UTR1 from lambda phage. The DNA may be linear or plasmid. Gene sequences may be engineered for cell-free expression in TXTL systems derived from the lysate source organism, such as: 5′rare codons for improved TXTL coupling, 5′ AT/GC content for improved TXTL coupling, UTR, RBS, termination sequences, 5′ fusions for improved TXTL coupling, gene fusions for improved TXTL coupling, fusions for protein stability, sequence deletions to promote solubility of membrane proteins, and protein tags.

mRNA may be supplied that utilizes translational components in the lysate and/or additions to the lysate to produce a protein. The mRNA may be from a purified natural source, or from a synthetically generated source, or can be generated in vitro, e.g., from an in-vitro transcription kit.

Non-canonical amino acids may be utilized in the composition. Non-canonical amino acids may be found naturally in the cellular-produced product, or may be artificially added to the product to produce desirable properties, such as tagging, visualization, resistance to degradation, or targeting. While implementation of non-canonical amino acids is difficult in cells, in cell-free systems implementation rates are higher due to the ability to saturate with the non-canonical amino acid. Examples, without limitation, of non-canonical amino acids, including ornithine, norleucine, homoarginine, tryptophan analogs, biphenylalanine, hydrolysine, pyrrolysine, or as described in (Blaskovich 2016) which is hereby incorporated by reference herein in its entirety.

Advantageously, any feature of a cell-free system may be analyzed and/or extracted by the predictive systems and methods of the invention. For example, any additive or energy recycling system may be analyzing for compatibility with the synthesis of a protein in a cell-free system.

Cell-free systems that may be used with the present invention are described in U.S. Pat. No. 11,004,536; U.S. Pat. Pub. No. 2020-0109429, U.S. Pat. Pub. No. 20200181670, U.S. Pat. Pub. No. U.S. Pat. Pub. No. 2022-0162651; PCT Pat. Pub. No. WO 2019/164558, and PCT Pat. Pub. No. WO 2021/147199, the entirety of the contents of each of which are incorporated herein by reference.

### Processor Implemented Systems

Methods of the present disclosure can be performed using software, hardware, firmware, hardwiring, or combinations of any of these. Features implementing functions can also be physically located at various positions, including being distributed such that portions of functions are implemented at different physical locations (e.g., imaging apparatus in one room and host workstation in another, or in separate buildings, for example, with wireless or wired connections).

Processors suitable for the execution of computer program include, by way of example, both general and special purpose microprocessors, and any one or more processor of any kind of digital computer. Generally, a processor will receive instructions and data from a read-only memory or a random access memory or both. Elements of computer are a processor for executing instructions and one or more memory devices for storing instructions and data. Generally, a computer will also include, or be operatively coupled to receive data from or transfer data to, or both, one or more non-transitory mass storage devices for storing data, e.g., magnetic, magneto-optical disks, or optical disks. In some embodiments, sensors on the system send process data via Bluetooth to a central data collection unit located outside of an incubator. In some embodiments, data is sent directly to the cloud rather than to physical storage devices. Information carriers suitable for embodying computer program instructions and data include all forms of non-volatile memory, including by way of example semiconductor memory devices, (e.g., EPROM, EEPROM, solid state drive (SSD), and flash memory devices); magnetic disks, (e.g., internal hard disks or removable disks); magneto-optical disks; and optical disks (e.g., CD and DVD disks). The processor and the memory can be supplemented by, or incorporated in, special purpose logic circuitry.

To provide for interaction with a user, the subject matter described herein can be implemented on a computer having an I/O device, e.g., a CRT, LCD, LED, or projection device for displaying information to the user and an input or output device such as a keyboard and a pointing device, (e.g., a mouse or a trackball), by which the user can provide input to the computer. Other kinds of devices can be used to provide for interaction with a user as well. For example, feedback provided to the user can be any form of sensory feedback (e.g., visual feedback, auditory feedback, or tactile feedback), and input from the user can be received in any form, including acoustic, speech, or tactile input.

The subject matter described herein can be implemented in a computing system that includes a back-end component (e.g., a data server), a middleware component (e.g., an application server), or a front-end component (e.g., a client computer having a graphical user interface or a web browser through which a user can interact with an implementation of the subject matter described herein), or any combination of such back-end, middleware, and front-end components. The components of the system can be interconnected through network by any form or medium of digital data communication, e.g., a communication network. Examples of communication networks include cell network (e.g., 3G, 4G, or 5G), a local area network (LAN), and a wide area network (WAN), e.g., the Internet.

The subject matter described herein can be implemented as one or more computer program products, such as one or more computer programs tangibly embodied in an information carrier (e.g., in a non-transitory computer-readable medium) for execution by, or to control the operation of, data processing apparatus (e.g., a programmable processor, a computer, or multiple computers). A computer program (also known as a program, software, software application, app, macro, or code) can be written in any form of programming language, including compiled or interpreted languages (e.g., C, C++, Perl), and it can be deployed in any form, including as a stand-alone program or as a module, component, subroutine, or other unit suitable for use in a computing environment. Systems and methods of the invention can include instructions written in any suitable programming language known in the art, including, without limitation, C, C++, Perl, Java, ActiveX, HTML5, Visual Basic, or JavaScript.

A computer program does not necessarily correspond to a file. A program can be stored in a file or a portion of file that holds other programs or data, in a single file dedicated to the program in question, or in multiple coordinated files (e.g., files that store one or more modules, sub-programs, or portions of code). A computer program can be deployed to be executed on one computer or on multiple computers at one site or distributed across multiple sites and interconnected by a communication network.

A file can be a digital file, for example, stored on a hard drive, SSD, CD, or other tangible, non-transitory medium. A file can be sent from one device to another over a network (e.g., as packets being sent from a server to a client, for example, through a Network Interface Card, modem, wireless card, or similar).

Writing a file according to embodiments of the invention involves transforming a tangible, non-transitory, computer-readable medium, for example, by adding, removing, or rearranging particles (e.g., with a net charge or dipole moment into patterns of magnetization by read/write heads), the patterns then representing new collocations of information about objective physical phenomena desired by, and useful to, the user. In some embodiments, writing involves a physical transformation of material in tangible, non-transitory computer readable media (e.g., with certain optical properties so that optical read/write devices can then read the new and useful collocation of information, e.g., burning a CD-ROM). In some embodiments, writing a file includes transforming a physical flash memory apparatus such as NAND flash memory device and storing information by transforming physical elements in an array of memory cells made from floating-gate transistors. Methods of writing a file are well-known in the art and, for example, can be invoked manually or automatically by a program or by a save command from software or a write command from a programming language.

Suitable computing devices typically include mass memory, at least one graphical user interface, at least one display device, and typically include communication between devices. The mass memory illustrates a type of computer-readable media, namely computer storage media.

Computer storage media may include volatile, nonvolatile, removable, and non-removable media implemented in any method or technology for storage of information, such as computer readable instructions, data structures, program modules, or other data. Examples of computer storage media include RAM, ROM, EEPROM, flash memory, or other memory technology, CD-ROM, digital versatile disks (DVD) or other optical storage, magnetic cassettes, magnetic tape, magnetic disk storage or other magnetic storage devices, Radiofrequency Identification (RFID) tags or chips, or any other medium which can be used to store the desired information and which can be accessed by a computing device.

As one skilled in the art would recognize as necessary or best-suited for performance of the methods of the invention, a computer system or machines employed in embodiments of the invention may include one or more processors (e.g., a central processing unit (CPU) a graphics processing unit (GPU) or both), a main memory and a static memory, which communicate with each other via a bus.

### Machine Learning Systems

Advantageously, the present invention also leverages machine learning systems, and in aspects of the invention the computing device may comprise a machine learning system.

Any suitable machine learning system may be used. For example, the machine learning systems may learn in a supervised manner, an unsupervised manner, a semi-supervised manner, or through reinforcement learning.

In supervised learning models, the machine learning system is given training data categorized as input variables paired with output variables from which to learn patterns and make inferences in order to generate a prediction on previously unseen test data. Supervised models replicate an identified mapping system and recognize and respond to patterns in data without explicit instructions. Supervised models are advantageous for performing classification tasks, in which data inputs are separated into categories. Supervised models are also advantageous for regression tasks, in which the output variable is a real value, such as a price or a volume. The accuracy of a supervised model is easy to evaluate, because there is a known output variable to which the model is optimizing.

In an unsupervised model or autonomous model, the machine learning system is only given input training data without paired output data from which to identify patterns autonomously. Unsupervised models identify underlying patterns or structures in training data to make predictions for test data. Unsupervised models are advantageous for clustering data, anomaly detection, and for independently discovering rules for data. The accuracy of unsupervised models is harder to evaluate because there is no predefined output variable to which the system is optimizing. Autonomous models may employ periods of both supervised and unsupervised learning in order to optimize predictions.

In semi-supervised models, the machine learning system is given training data comprising input variables, with output variable pairs available for only a limited pool of the input variables. The model uses the input variables with known output variables and the remaining input training data to learn patterns and make inferences in order to generate a prediction on previously unseen test data. A semi-supervised model may query the user for additional paired output data based on unlabeled data.

In a reinforcement learning model, the machine learning system is given neither input variables nor output variables. Rather, the model provides a “reward” condition and then seeks to maximize the cumulative reward condition by trial and error. A common reinforcement learning model is a Markov Decision Process.

A common supervised learning model is a “decision tree.” Decision trees are non-parametric supervised learning models that use simple decision rules to infer a classification for test data from the features in the test data. In classification trees, test data take a finite set of values, or classes, whereas in regression trees, the test data can take continuous values, such as real numbers. Decision trees have some advantages in that they are simple to understand and can be visualized as a tree starting at the root (usually a single node) and repeatedly branch to the leaves (multiple nodes) that are associated with the classification. See Criminisi, 2012, Decision Forests: A unified framework for classification, regression, density estimation, manifold learning and semi-supervised learning, Foundations and Trends in Computer Graphics and Vision 7(2-3):81-227, incorporated by reference.

Another supervised learning model is a “support-vector machine” (SVM) or “support-vector network.” SVMs are supervised learning models for classification and regression problems. When used for classification of new data into one of two categories, such as having a disease or not having a disease, an SVM creates a hyperplane in multidimensional space that separates data points into one category or the other. Although the original problem may be expressed in terms that require only finite dimensional space, linear separation of data between categories may not be possible in finite dimensional space. Consequently, multidimensional space is selected to allow construction of hyperplanes that afford clean separation of data points. See Press, W. H. et al., Section 16.5. Support Vector Machines. Numerical Recipes: The Art of Scientific Computing (3rd ed.). New York: Cambridge University (2007), incorporated herein by reference. Where output variables are unavailable for input variables in the training data, SVMs can be designed as unsupervised learning models using support vector clustering. See Ben-Hur, 2001, Support Vector Clustering, J Mach Learning Res 2:125-137, incorporated by reference.

Some models rely on clustering training data and test data to find patterns and make predictions. A “k-nearest neighbor” (k-NN) model is a non-parametric supervised learning model for classification and regression problems. A k-nearest neighbor model assumes that similar data exists in close proximity, and assigns a category or value to each data point based on the k nearest data points. k-NN models may be advantageous when the data has few outliers and can be defined by homogeneous features. A common unsupervised learning model that uses clustering is a “k-means” clustering model. A k-means model looks to find clusters of data in input data and test data. K-means models are advantageous when a defined number of clusters are known to exist in the data and are also advantageous when the test data has few outliers and can be defined homogeneous features. Additional models that cluster training data include, for example, farthest-neighbor, centroid, sum-of-squares, fuzzy k-means, and Jarvis-Patrick clustering.

Bayesian algorithms can also be used to find patterns in training and test data to make predictions. Bayesian networks are probabilistic graphical models that represent a set of random variables and their conditional dependencies via directed acyclic graphs (DAGs). The DAGs have nodes that represent random variables that may be observable quantities, latent variables, node unknown parameters or hypotheses. Edges represent conditional dependencies; nodes that are not connected represent variables that are conditionally independent of each other. Each is associated with a probability function that takes, as input, a particular set of values for the node's parent variables, and gives (as output) the probability (or probability distribution, if applicable) of the variable represented by the node.

Regression analysis is another statistical process that can be used to find patterns in training and test data to make predictions. It includes techniques for modeling and analyzing relationships between a multiple variables. Specifically, regression analysis focuses on changes in a dependent variable in response to changes in single independent variables. Regression analysis can be used to estimate the conditional expectation of the dependent variable given the independent variables. The variation of the dependent variable may be characterized around a regression function and described by a probability distribution. Parameters of the regression model may be estimated using, for example, least squares methods, Bayesian methods, percentage regression, least absolute deviations, nonparametric regression, or distance metric learning.

Trained machine learning models can become “stable learners.” A stable learner is a model that is less sensitive to perturbation of predictions based on new training data. Stable learners can be advantageous where test data is stable, but can be less advantageous where the system needs to continually improve performance to accurately predict new test data.

Several machine learning system types can be combined into a final predictive models known as ensembles. Ensembles can be divided into two types, homogenous ensembles and heterogeneous ensembles. Homogenous ensembles combine multiple machine learning models of the same type. Heterogeneous ensembles combine multiple machine learning models of different types. Ensembles can provide the advantage of being more accurate than any of the individual member models (“members”) in the ensemble. The number of members combined in an ensemble may impact the accuracy of a final prediction. Accordingly, it is advantageous to determine the optimal number of members when designing an ensemble system.

Ensembles may combine or aggregate outputs from individual members using “voting”-type methods for classification systems and “averaging”-type methods for regression systems.

In a “majority voting” method, each member makes a prediction for test data and the prediction that receives more than half of the votes is the final output for the ensemble. If none of the predictions receives more than half of the votes, it may be determined that the ensemble is unable to make a stable prediction. In a “plurality voting” method the most voted prediction, even if receiving less than half of the votes, may be considered the final output for the ensemble. In a “weighted voting” method, the votes of more accurate members are multiplied by a weight afforded each member based on its accuracy.

In a “simple averaging” method, each member makes a prediction for test data and the average of the outputs is calculated. This method reduces overfit and can be advantageous in creating smoother regression models. In a “weight averaging” method, the prediction output of each member is multiplied by a weight afforded each member based on its accuracy. Voting methods, averaging methods, and weighted methods can be combined to improve the accuracy of ensembles.

Members within an ensemble can each be trained independently or new members can be trained utilizing information from previously trained members. In a “parallel ensemble”, the ensemble seeks to provide greater accuracy than individual members by exploiting the independence between members, for example, by training multiple members simultaneously and aggregating the outputs from members. In “sequential ensemble systems”, the ensemble seeks to provide greater accuracy than individual members by exploiting the dependence between members, for example, by utilizing information from a first member to improve the training of a second member and weighting outputs from members.

Overall accuracy for ensembles can also be optimized by using ensemble meta-algorithms, for example a “bagging” algorithm to reduce variance, a “boosting” algorithm to reduce bias, or a “stacking” algorithm to improve predictions.

Boosting algorithms reduce bias and can be used to improve less accurate, or “weak learning” models. A member may be considered a “weak learning” model if it has a substantial error rate, but its performance is non-random, for example an error rate of 0.5 for binary classifications. Boosting algorithms incrementally build the ensemble by training each member sequentially with the same training data set, examining prediction errors for test data, and assigning weights to training data based on the difficulty for members to make an accurate prediction. In each sequential member trained, the algorithm emphasizes training data that previous members found difficult. Members are then weighted based on the accuracy of their prediction outputs in view of the weight applied to their training data. The predictions from each member may be combined by weighted voting-type or weighted averaging-type methods. Boosting algorithms are advantageous when combining multiple weak learning models. Boosting algorithms may, however, result in over-fitting test data to training data.

Examples of boosting algorithms include AdaBoost, gradient boosting, eXtreme Gradient Boost (XGBoost). See Freund, 1997, A decision-theoretic generalization of on-line learning and an application to boosting, J Comp Sys Sci 55:119; and Chen, 2016, XGBoost: A Scalable Tree Boosting System, arXiv: 1603.02754, both incorporated by reference.

Bagging algorithms or “bootstrap aggregation” algorithms reduce variance by averaging together multiple estimates from members. Bagging algorithms provide each member with a random sub-sample of a full training data set, with each random sub-sample known as a “bootstrap” sample. In the bootstrap samples, some data from the training data set may appear more than once and some data from the training data set may not be present. Because sub-samples can be generated independently from one another, training can be done in parallel. The predictions for test data from each member are then aggregated, such as by voting-type or averaging-type methods.

An example of a bagging algorithm that may be utilized is a “random forests”. In a random forest the ensemble combines multiple randomized decision tree models. Each decision tree model is trained from a bootstrap sample from a training set. The training set itself may be a random subset of features from an even larger training set. By providing a random subset of the larger training set at each split in the learning process, spurious correlations that can results from the presence of individual features that are strong predictors for the response variable are reduced. By averaging predictions for test data, variance of the ensemble decreases resulting in an improved prediction. Random forests may autonomous models and may include periods of both supervised and unsupervised learning. Bagging may be less advantageous in optimizing an ensemble combining stable learning systems, since stable learning systems tend provide generalized outputs with less variability over the bootstrap samples. See Breiman, 2001, Random Forests, Machine Learning 45:5-32, incorporated by reference.

Stacking algorithms or “stacked generalization” algorithms improve predictions by using a meta-machine learning model to combine and build the ensemble. In stacking algorithms, base member models are trained with a training dataset and generate as an output a new dataset. This new dataset is then used as a training dataset for the meta-machine learning model to build the ensemble. Stacking algorithms are generally advantageous when building heterogeneous ensembles.

Neural networks, modeled on the human brain, allow for processing of information and machine learning. Neural networks include nodes that mimic the function of individual neurons, and the nodes are organized into layers. Neural networks include an input layer, an output layer, and one or more hidden layers that define connections from the input layer to the output layer.

Systems and methods of the invention may include any neural network that facilitates machine learning. The system may include a known neural network architecture, such as GoogleNet (Szegedy, et al. Going deeper with convolutions, in CVPR 2015, 2015); AlexNet (Krizhevsky, et al. Imagenet classification with deep convolutional neural networks, in Pereira, et al. Eds., Advances in Neural Information Processing Systems 25, pages 1097-3105, Curran Associates, Inc., 2012); VGG16 (Simonyan & Zisserman, Very deep convolutional networks for large-scale image recognition, CoRR, abs/3409.1556, 2014); or FaceNet (Wang et al., Face Search at Scale: 80 Million Gallery, 2015), each of the aforementioned references are incorporated by reference.

Deep learning neural networks (also known as deep structured learning, hierarchical learning or deep machine learning) include a class of machine learning operations that use a cascade of many layers of nonlinear processing units for feature extraction and transformation.

Each successive layer uses the output from the previous layer as input. The algorithms may be supervised or unsupervised and applications include pattern analysis (unsupervised) and classification (supervised). Certain embodiments are based on unsupervised learning of multiple levels of features or representations of the data. Higher level features are derived from lower level features to form a hierarchical representation. Those features are preferably represented within nodes as feature vectors. Deep learning by the neural network includes learning multiple levels of representations that correspond to different levels of abstraction; the levels form a hierarchy of concepts. In some embodiments, the neural network includes at least 5 and preferably more than ten hidden layers. The many layers between the input and the output allow the system to operate via multiple processing layers.

Within the network, nodes are connected in layers, and signals travel from the input layer to the output layer. Each node in the input layer may correspond to a respective one of the features from the training data. The nodes of the hidden layer are calculated as a function of a bias term and a weighted sum of the nodes of the input layer, where a respective weight is assigned to each connection between a node of the input layer and a node in the hidden layer. The bias term and the weights between the input layer and the hidden layer are learned autonomously in the training of the neural network. The network may include thousands or millions of nodes and connections. Typically, the signals and state of artificial neurons are real numbers, typically between 0 and 1. Optionally, there may be a threshold function or limiting function on each connection and on the unit itself, such that the signal must surpass the limit before propagating. Back propagation is the use of forward stimulation to modify connection weights, and is sometimes done to train the network using known correct outputs. See WO 2016/182551, U.S. Pub. 2016/0174902, U.S. Pat. No. 8,639,043, and U.S. Pub. 2017/0053398, each incorporated by reference.

Deep learning is part of a broader family of machine learning methods based on learning representations of data. An observation can be represented in many ways such as a vector of intensity values per pixel, or in a more abstract way as a set of edges, regions of particular shape, etc. Those features are represented at nodes in the network. Preferably, each feature is structured as a feature vector, a multi-dimensional vector of numerical features that represent some object. The feature provides a numerical representation of objects, since such representations facilitate processing and statistical analysis. Feature vectors are similar to the vectors of explanatory variables used in statistical procedures such as linear regression. Feature vectors are often combined with weights using a dot product in order to construct a linear predictor function that is used to determine a score for making a prediction.

The vector space associated with those vectors may be referred to as the feature space. In order to reduce the dimensionality of the feature space, dimensionality reduction may be employed. Higher-level features can be obtained from already available features and added to the feature vector, in a process referred to as feature construction. Feature construction is the application of a set of constructive operators to a set of existing features resulting in construction of new features.

For example, a convolutional neural network (CNN) is a class of deep neural network generally designed for two-dimensional image inputs in which a signal travels from the input layer through hidden layers comprising “convolutional layers” and “fully connected layers” to the output layer. In the input layer, each pixel from a signal is mapped to a node. The input layer is connected to a convolutional layer. In a convolutional layer, each node is “sparsely connected”, that is connected to only a sub-matrix of nodes from the previous layer. The connection between the submatrix of nodes and the convolutional layer is subject to a bias term as a set of weights designed detect a given feature in the input. The submatrix and weights together are known as a “filter,” “kernel,” or “feature detector”. For a given convolutional layer, each filter is the same size and shape and applies the same set of weights. Each node in the convolutional layer is provided a summary of the weighted information from the filter as a scalar dot product. The filters are staggered from one another and may overlap such that each node in convolution layer provides a weighted summary for a different sub-matrix from the previous layer. A threshold function may be applied to each node in the convolution layer to determine whether the node will propagate the information from the filter, a function known as “squashing.”

Sliding the filter systematically across the entire input allows the filter to discover a given feature anywhere in the input. The function of sliding the filter over entire image can be controlled by the number of nodes over which the filter movies, known as the “stride” of the convolutional layer. The stride determines the distance that each filter is staggered from adjacent filters and the degree of overlap between filters. The final two-dimensional array of dot products of the convolutional layer is known as the “convolved feature,” “activation map,” or “feature map.”

Filters may also have a given depth. For example, color images have multiple channels, typically one for each color channel, such as red, green, and blue. This means that a single color image provided as an input to the input layer is, in fact, three images. A filter must always have the same number of channels as the input, referred to as “depth”. If an input image has 3 channels (e.g. a depth of 3), then a filter applied to that image must also have 3 channels (e.g. a depth of 3). In this case, a 3×3 filter would in fact be 3×3×3 or [3, 3, 3] for rows, columns, and depth. Regardless of the depth of the input and depth of the filter, the filter is applied to the input using a dot product operation which results in a single value. This means that if a convolutional layer has 32 filters, these 32 filters are not just two-dimensional for the two-dimensional image input, but are also three-dimensional, having specific filter weights for each of the three channels. Each filter results in a single feature map.

Different filters produce different feature maps. A convolutional layer may apply a different filter depending on the given input, with the types of filters available learned during training of the network. For example, the network may be trained to apply filters for a specific task the network is trained to resolve, such as detecting whether an input image contains a vertical line. The convolution layer may be trained to apply any number of possible filters to an input image, for example from 32 to 512 filters.

In some instances it may also be convenient to “pad” an input to a convolutional layer with zero values around the border of the input, a process known as zero-padding. Zero-padding allows the size of feature maps to be controlled. This can allow for the feature map to remain the same size as the input through multiple layers of the CNN. The function of adding zero-padding is known as “wide-convolution” versus “narrow convolution” when no zero-padding is added.

The use of multiple convolutional layers in the network allows for hierarchical decomposition of the input. Convolutional filters that operate directly on input values may learn to extract low level features, such as lines. Convolutional filters that operate on the output from earlier convolution layers may learn to extract features that are combinations of lower-level features, such as features that comprise multiple lines to express shapes.

A CNN may also comprise nonlinear layers (ReLU) and/or pooling or sub sampling layers. A ReLU layer receives a feature map and replaces any negative values in the feature map with a zero. The purpose of the ReLU layer is to introduce non-linearity into the CNN and is advantageous when the input data that the CNN is expected to learn and identify is non-linear. The non-linear output map from a ReLU is known as a “rectified” feature map. A pooling layer reduces the size of the feature map or rectified feature map through dimensionality reduction in a process known as “spatial pooling,” “subsampling,” or downsampling.” For example, each node in a pooling layer may be connected to a sub-matrix of nodes from a convolution or ReLU layer. Each node in the pooling layer may then provide, for example, only the highest value, average of, or sum of the values in each submatrix. Pooling layers can be advantageous to make input representations smaller and more manageable, reduce the number of parameters and computations in the network, reduce the impact of distortions in the input image, and help scale representation of the image. This may reduce training time and control overfitting in the CNN.

The final output from the convolutional, ReLU, and/or pooling layers, is provided to a fully connected layer. The fully connected layers operate under the same principles as a traditional neural network. In a fully connected layer each node in the layer is connected to all of the nodes in a previous layer and all of the nodes in a succeeding layer. The purpose of a fully connected layer is to classify the features extracted by the convolutional layers, for example using single vector machines (SVM).

Backpropagation in CCNs involves adjusting the weights of filters based on the error rate of the CNN, known as “loss.” During backpropagation, the CNN determines the estimated loss at every node in each convolutional layer and adjusts filter weights accordingly to minimize loss. A CNN may be trained by multiple rounds of backpropagation.

A deconvolutional neural network (DNN) is another class of deep neural network designed to generate an image from a feature map or from the output from a CNN. A DNN learns and makes predictions as to the pooling, ReLU, and convolution layers that a feature map may have undergone and performs the opposite function, e.g. unpooling and deconvolution.

### EXPERIMENTAL EXAMPLES

### Feature Extraction

Systems and methods of the invention provide a score of synthesis difficulty based on known limitations from cell-free systems. The systems and methods benefit from the extraction of features correlated with successfully protein synthesis on cell-free systems.

Accordingly, in a first step, 970 proteins for which protein data was available were correlated by the system to actual synthesis success. 2 buckets were employed, “Standard” and “Risk Factors Present.” Standard is an amalgamation of factors with a score of 0.75 or higher, including “Standard Risk”, “Low Risk”, and “Moderate Risk.” “Risk Factors” is an amalgamation of factors with a score of lower than 0.75, including “High Risk” and “Rejects.” 952 proteins fell within the Standard group and 12 proteins fell within the Risk Factor Group.

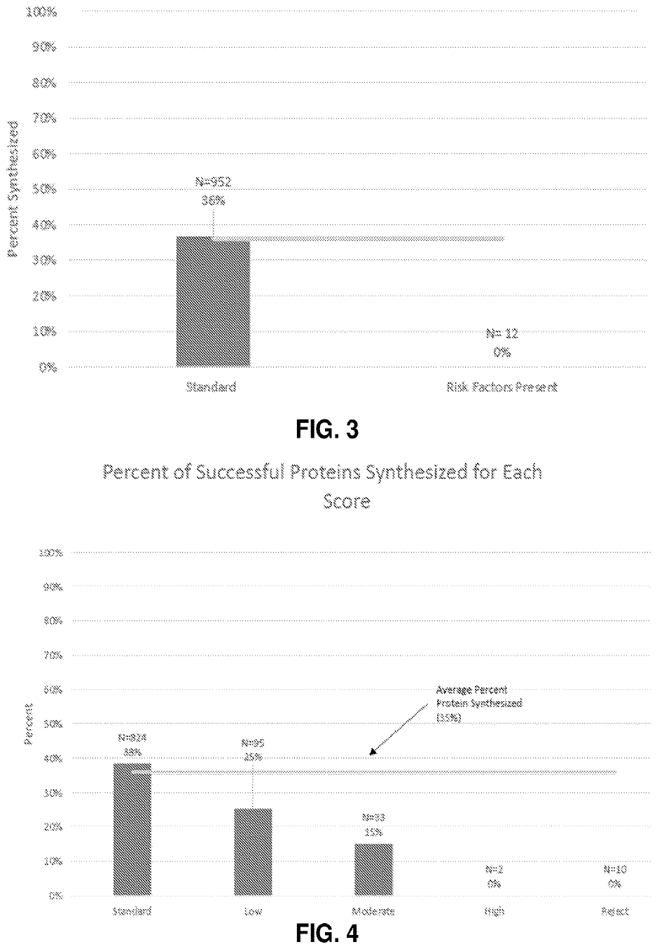

FIG. 3 is a graph of percent protein synthesis outcomes for the Standard and Risk Factor groups. 36% of the proteins in the Standard group were successfully synthesized. 0% of the proteins in the Risk Factor group were synthesized.

FIG. 4 is a graph of protein synthesis outcomes for each prediction group. Out of the 345 proteins successfully synthesized, 38% of “Standard Risk” proteins were synthesized, 25% of “Low Risk” proteins were synthesized, and 15% of “Moderate Risk” proteins were synthesized. Given the lower synthesis rate, “Moderate Risk” proteins were further analyzed.

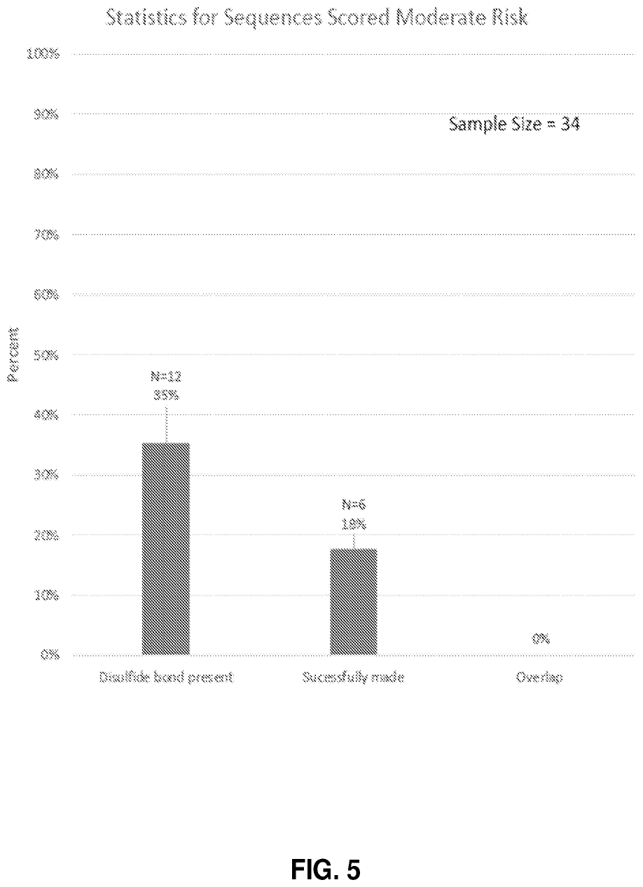

FIG. 5 is a graph of protein synthesis outcomes for moderate risk proteins. “Moderate Risk” proteins were divided into two groups which had no overlap, those proteins with a disulfide bond present (n=12) and those with no disulfide bonds present (n=6). All of the proteins with disulfide bonds present in this group were successfully synthesized and none of the proteins with no disulfide bonds present were synthesized. This feature was extracted from the data for further testing.

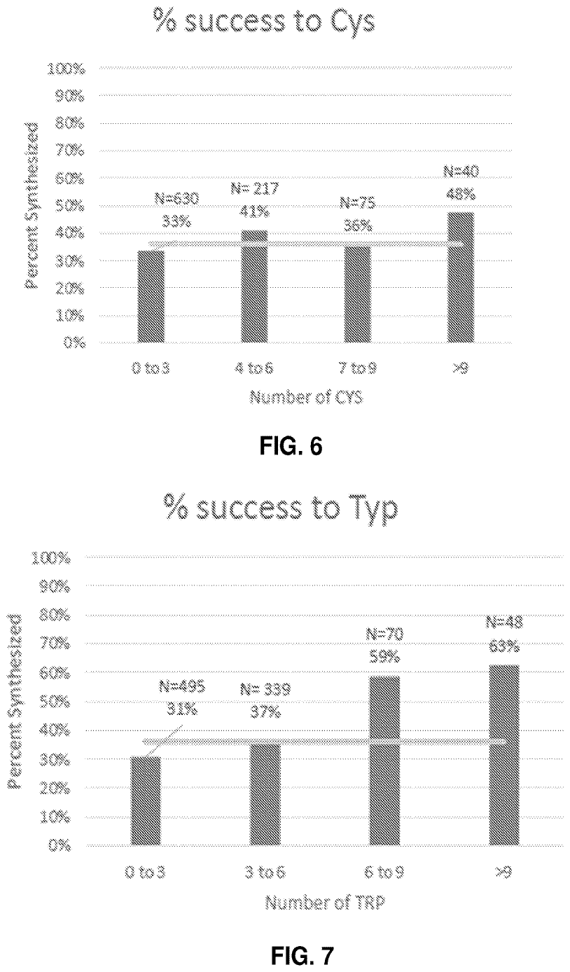

FIG. 6 is a graph of protein synthesis outcomes based on Cystine (Cys) residues.

FIG. 7 is a graph of protein synthesis outcomes based on Tryptophan (Typ).

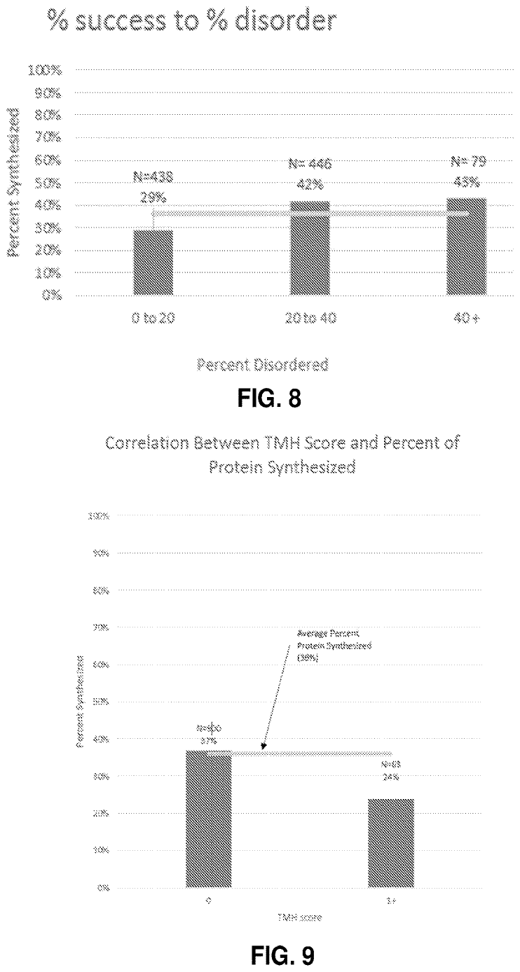

FIG. 8 is a graph of protein synthesis outcomes based on disordered percentage.

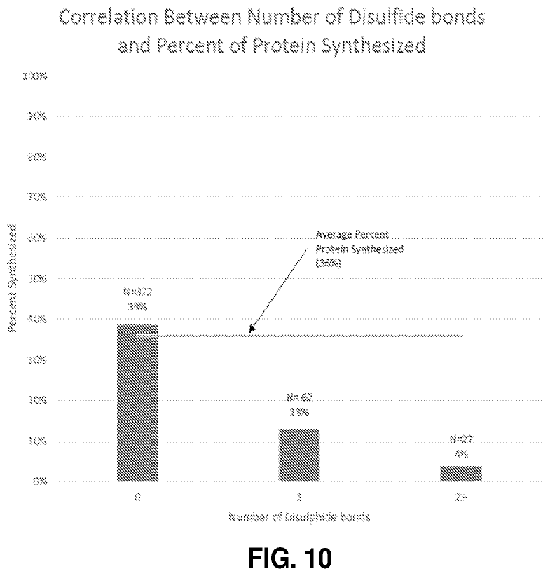

FIG. 9 is a graph of protein synthesis outcomes based on transmembrane helices (TMH).

FIG. 10 is a graph of protein synthesis outcomes based on the number of disulfide bonds.

Two parameters were bioinformatically predicted to correlated to higher synthesis risk: disulfide bonds (26% success rate reduction) and transmembrane domains (13% success rate reduction). This data was fed into the system and processed.

### Score Prediction

In a second step, scoring is provided a gauge of success instead of risk (e, g. rule-in, rather than rule-out). Empirical and combinatoric cell-free expression is used to conduct synthesis reactions. Additional protein synthesis data, correlations to external data streams such as protein domain classes and reports from literature, and data from multiple conditions are empirically tested. The cell free systems allow hundreds of thousands of synthesis reactions to be conducted to guarantee production (or not) based on protein sequence alone. Multiple systems (e.g., Bacillus, Pichia, Tobacco, Wheat Germ, and equivalent plant and eukaryotes) are tested and multiple conditions (e.g. time, temperature, DNA tags, etc.) are tested. The combinatoric results in the same closed-loop format are used to empirically tie out factors that predict protein synthesis success. Success is judged by a metric of percent protein synthesis success. Sequence information is collected across platform runs, and public knowledge is also used to improve success rates from sequence to manufacturability.

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### Equivalents

While the present invention has been described in conjunction with certain embodiments, one of ordinary skill, after reading the foregoing specification, will be able to effect various changes, substitutions of equivalents, and other alterations to the compositions and methods set forth herein.

1. A system for determining the probability of cell-free synthesis of a protein, the system comprising:
a computing device comprising a hardware processor coupled to memory containing instructions executable by the processor to cause the system to:
receive data comprising the properties of a protein to be synthesized;
connect to one or more databases comprising protein synthesis data;
calculate the probability of success for the protein to be synthesized;
provide the calculated probability of success for the protein to be synthesized.

2. The system of claim 1, wherein the one or more databases includes protein synthesis data for proteins previously processed through the system.

3. The system of claim 1, wherein the calculating step comprises analyzing the properties of the protein to be synthesized.

4. The system of claim 3, wherein the properties of the protein for analysis are selected from a group consisting of: (i) transmembrane helices, (ii) disulfide bonds, (iii) tryptophan residues, (iv) cysteine residues, (v) percent disorder, (vi) protein size, and (vii) any combination thereof.

5. The system of claim 1, wherein the system is a closed loop-system.

6. The system of claim 1, wherein the system is adapted to update the one or more databases to include protein synthesis data for proteins processed through the system.

7. The system of claim 1, wherein the system iteratively updates the calculating step upon receiving additional protein synthesis data.

8. The system of claim 1, wherein the one or more databases comprise publicly available databases comprising protein structure and/or synthesis information.

9. The system of claim 8, wherein the publicly available information platforms may be selected from a group consisting of: Protein Data Bank (PDB), National Library of Medicine (NLM), GenBank, Reference Sequence (RefSeq), UniProt, AlphaFold, or Expasy.

10. The system of claim 1, wherein the system further analyzes information regarding reaction conditions for the protein to be synthesized to achieve the highest probability of success for protein synthesis based on the use of different reaction conditions.

11. The system of claim 10, wherein the reaction conditions analyzed are selected from a group consisting of: (i) species for cell lysate to be used for in vitro synthesis, (ii) supplements for the in vitro synthesis, (iii) reaction temperature, (iv) duration of the reaction, (v) pH, (vi) solvents, (vii) technique for purification of synthesized protein, (viii) reagents for purification, (ix) concentration of DNA for in vitro synthesis, and (x) any combination thereof.

12. The system of claim 1, wherein the system further provides recommendations for editing the sequence of the protein to be synthesized to increase the likelihood of success of synthesizing the protein or an analog to the protein.

13. The system of claim 1, wherein the system further assigns a protein family for the protein to be synthesized.

14. The system of claim 13, wherein the protein family assignment is based on the data available on Pfam database.

15. The system of claim 1, wherein the probability for cell-free synthesis of the protein is provided as standard, low risk, moderate risk, high risk, or unlikely.

16. The system of claim 1, wherein the probability for cell-free synthesis of the protein is provided quantitatively as a percentage, wherein 100% represents a high likelihood of success and 0% represents low likelihood of success.

17. The system of claims 1, wherein the computing device comprises a machine learning system.

18. The system of claim 17, wherein the machine learning system comprises a neural network.

19. A method for determining the probability of cell-free synthesis of a protein, the method comprising:
receiving data comprising the properties of a protein to be synthesized;
connecting to one or more databases comprising protein synthesis data;
calculating the probability of success for the protein to be synthesized;
providing the calculated probability of success for the protein to be synthesized.

20. The method of claim 19, wherein the one or more databases includes protein synthesis data for proteins previously analyzed with the method.

21. The method of claim 19, wherein the calculating step comprises analyzing the properties of the protein to be synthesized.

22. The method of claim 21, wherein the properties of the protein that are analyzed are selected from a group consisting of: (i) transmembrane helices, (ii) disulfide bonds, (iii) tryptophan residues, (iv) cysteine residues, (v) percent disorder, (vi) protein size, and (vii) any combination thereof.

23. The method of claim 19, wherein the method comprises updating the one or more databases to include protein synthesis data for proteins previously analyzed by the method.

24. The method of claim 19, wherein the method comprises iteratively updating the calculating step upon receiving additional protein synthesis data.

25. The method of claim 19, wherein the one or more databases comprise publicly available information platforms comprising protein structure and/or synthesis information.

26. The method of claim 25, wherein the publicly available information platforms may be selected from a group consisting of: Protein Data Bank (PDB), National Library of Medicine (NLM), GenBank, Reference Sequence (RefSeq), UniProt, or Expasy.

27. The method of claim 19, wherein the method further comprises analyzing information regarding reaction conditions for the protein to be synthesized to achieve the highest probability of success for protein synthesis based on the use of different reaction conditions.

28. The method of claim 27, wherein the reaction conditions analyzed are selected from a group consisting of: (i) species for cell lysate to be used for in vitro synthesis, (ii) supplements for the in vitro synthesis, (iii) reaction temperature, (iv) duration of the reaction, (v) pH, (vi) solvents, (vii) technique for purification of synthesized protein, (viii) reagents for purification, (ix) concentration of DNA for in vitro synthesis, and (x) any combination thereof.

29. The method of claim 19, wherein the method further comprises providing recommendations for editing the sequence of the protein to be synthesized to increase the likelihood of success of synthesizing the protein or an analog to the protein.

30. The method of claim 19, wherein the method further comprises assigning a protein family for the protein to be synthesized.

31. The method of claim 30, wherein the protein family assignment is based on the data available on Pfam database.

32. The method of claim 19, wherein the probability for cell-free synthesis of protein is provided as standard, low risk, moderate risk, high risk, or unlikely.

33. The method of claim 19, wherein the probability for cell-free synthesis of protein is provided quantitatively as percentage, wherein 100% represents a high likelihood of success and 0% represents low likelihood of success.

34. The method of claims 19, wherein the computing device comprises a machine learning system.

35. The system of claim 34, wherein the machine learning system comprises a neural network.
